(19) United States
(12) Patent Application Publication
(10) Pub. No.: US 2022/0257067 A1
(43) Pub. Date: Aug. 18, 2022

(54) Hand Sanitizing Station Apparatus

(71) Applicant: Robert Pineau, Burlington (CA)

(72) Inventor: Robert Pineau, Burlington (CA)

(21) Appl. No.: 17/177,829

(22) Filed: Feb. 17, 2021

(51) Int. Cl. A47K 5/12; A61L 2/26; A61L 2/00

(52) U.S. Cl. CPC A47K 5/1217; A47K 5/1211; A61L 2202/15; A61L 2/0082; A61L 2202/14; A61L 2/26

(57) ABSTRACT

A hand sanitizing station apparatus for convenient zero contact sanitization includes a basin coupled to a housing. A misting tip is coupled to the basin through a tip aperture. A head of the misting tip has a release aperture to create a mist. A feeder tube is coupled to the misting tip and a tank is coupled to the feeder tube and contains pressurized hand sanitizer. A release valve is coupled to the feeder tube between the tank and the misting tip. A motion sensor is coupled to the basin and is in operational communication with the release valve to allow flow of the pressurized hand sanitizer through to the release aperture of the misting tip when a user's hands are placed within the basin.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention.

The disclosure relates to hand sanitizing devices and more particularly pertains to a new hand sanitizing device for convenient zero contact sanitization. The present device includes a basin to capture excess sanitizer, as well as a motion activated misting tip to release pressurized sanitizer.

### (2) Description of Related Art including information disclosed under 37 CFR 1.97 and 1.98.

The prior art relates to hand sanitizing devices. Known devices include contactless sanitizer dispensers that release a liquid or foam down onto the user's hands. These devices often create a mess on the floor or catch platform beneath the dispenser. Existing devices lack a misting tip to create an even distribution of pressurized sanitizer and instead typically release a concentrated liquid or foam.

### BRIEF SUMMARY OF THE INVENTION

An embodiment of the disclosure meets the needs presented above by generally comprising a housing having a housing front side, a housing back side, a housing top side, a housing bottom side, a housing left side, and a housing right side defining a housing inside. A basin is coupled to the housing. The basin is coupled to the housing top side and has a basin front side, a basin back side, a basin left side, a basin right side, a basin bottom side, and a basin rim. The basin bottom side has a tip aperture extending therethrough. A misting tip is coupled to the basin. The misting tip has a stem extending through the tip aperture and a head coupled to the stem above the basin bottom side. The head has a release aperture to create a mist. A feeder tube is coupled to the misting tip. The feeder tube is in fluid communication with the stem of the misting tip. A tank is coupled to the feeder tube and contains pressurized hand sanitizer. A release valve is coupled to the feeder tube between the tank and the misting tip. A motion sensor is coupled to the basin. The motion sensor is in operational communication with the release valve to allow flow of the pressurized hand sanitizer through to the release aperture of the misting tip when a user's hands are placed within the basin.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a hand sanitizing station apparatus according to an embodiment of the disclosure.

FIG. 2 is a cross-sectional view of an embodiment of the disclosure along the line 1-1 of FIG. 1.

FIG. 3 is a rear elevation view of an embodiment of the disclosure.

FIG. 4 is an isometric view of an embodiment of the disclosure.

FIG. 5 is a side elevation view of an embodiment of the disclosure.

FIG. 6 is an isometric view of an embodiment of the disclosure.

### DETAILED DESCRIPTION OF THE INVENTION

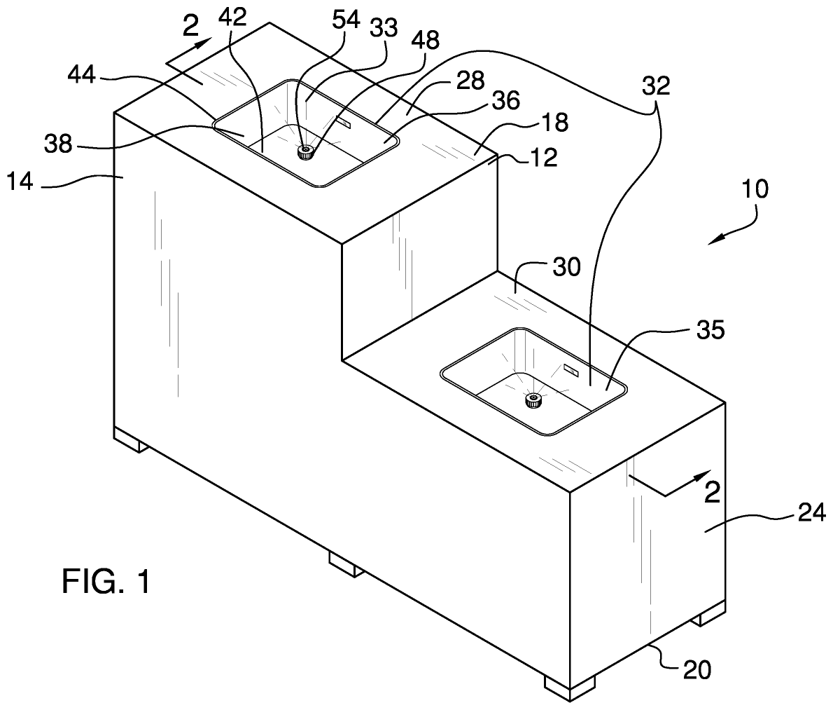

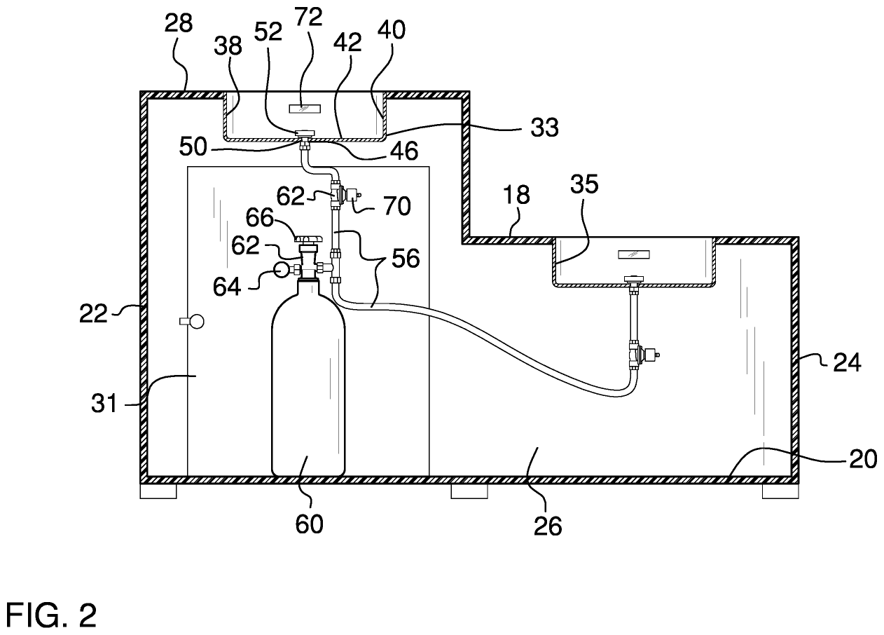

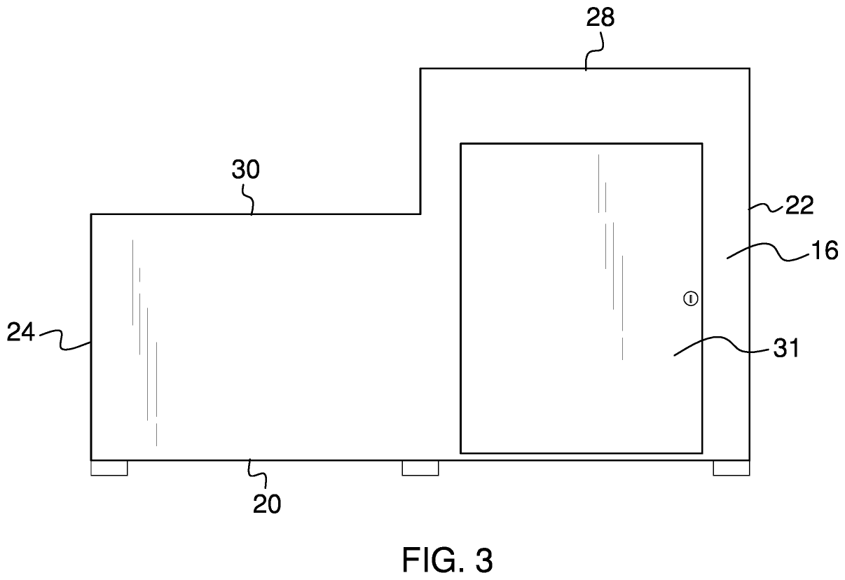

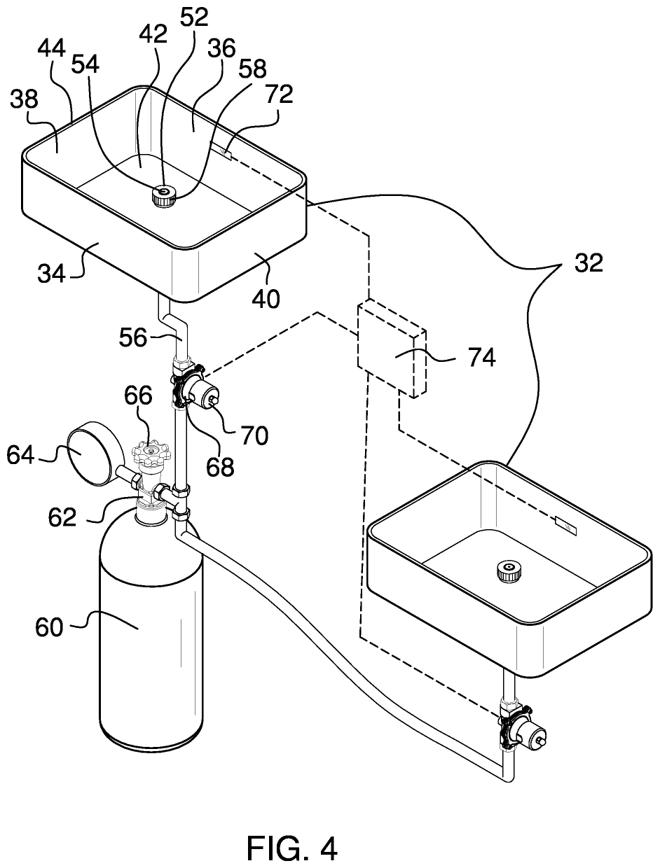

With reference now to the drawings, and in particular to FIGS. 1 through 6 thereof, a new hand sanitizing device embodying the principles and concepts of an embodiment of the disclosure and generally designated by the reference numeral 10 will be described.

As best illustrated in FIGS. 1 through 6, the hand sanitizing station apparatus 10 generally comprises a housing 12 having a housing front side 14, a housing back side 16, a housing top side 18, a housing bottom side 20, a housing left side 22, and a housing right side 24 defining a housing inside 26. The housing top side 18 may have an upper tier 28 and a lower tier 30. The housing back side 16 may have an access door 31 to provide access to the housing inside 26.

A pair of basins 32 is coupled to the housing 12 and includes an upper basin 33 and a lower basin 35 coupled to the upper tier 28 and the lower tier 30, respectively. Each basin 32 has a basin front side 34, a basin back side 36, a basin left side 38, a basin right side 40, a basin bottom side 42, and a basin rim 44. The basin rim 44 may rest flush with the housing top side 18. The basin bottom side 20 has a tip aperture 46 extending therethrough. Each basin 32 may have rounded corners for ease of cleaning.

A pair of misting tips 48 is coupled to the pair of basins 32. Each misting tip 48 has a stem 50 extending through the tip aperture 46 and a head 52 coupled to the stem 50 above the basin bottom side 20. The head 52 has a release aperture 54 to create a mist. A pair of feeder tubes 56 is coupled to the pair of misting tips 48. The feeder tube 56 is in fluid communication with the stem 50 of the misting tip. The head 52 may be cylindrical with a ridged edge 58 to allow for ease of installation and removal.

A tank 60 is coupled to the feeder tube 56. The tank 60 contains pressurized hand sanitizer and may be coupled within the housing inside 26. A pressure regulator 62 is coupled to the tank 60 and may include a pressure gauge 64 and a spurred handle 66 to adjust the pressure of fluid released by the tank 60. A pair of release valves 68 is coupled to the pair of feeder tubes 56. Each release valve 68 is coupled between the tank 60 and the respective misting tip 48. The release valve 68 may function with a solenoid 70.

A pair of motion sensors 72 is coupled to the pair of basins 32. Each motion sensor 72 is in operational communication with the respective release valve 68 to allow flow of the pressurized hand sanitizer through to the release aperture 54 of the respective misting tip when a user's hands are placed within the respective basin 32. The motion sensor 72 may be coupled to the basin back side 36. A power source 74 is in operational communication with the pair of motion sensors 72 and the pair of release valves 68.

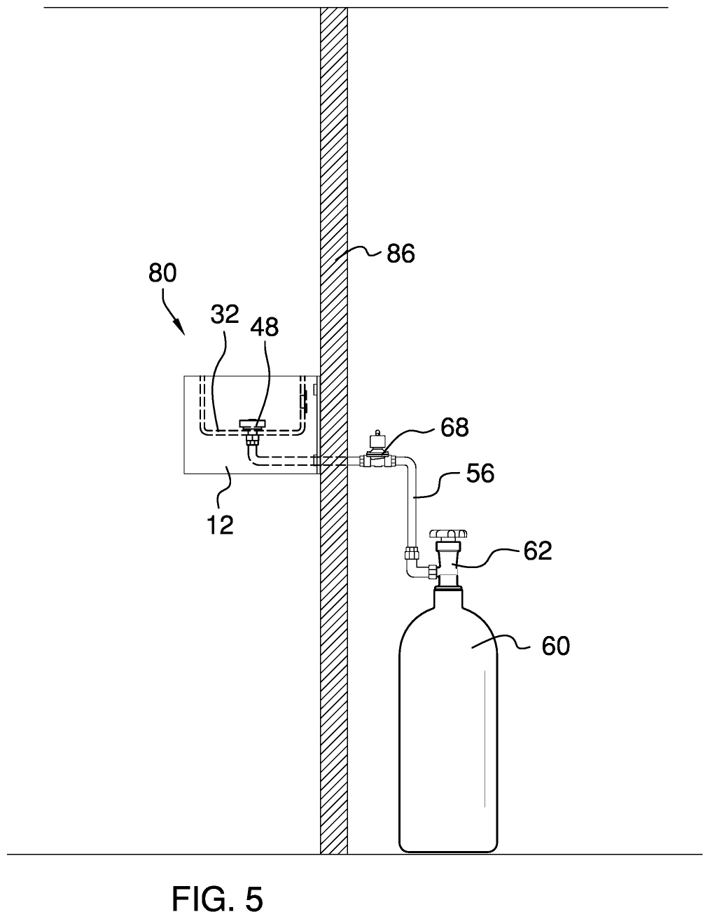

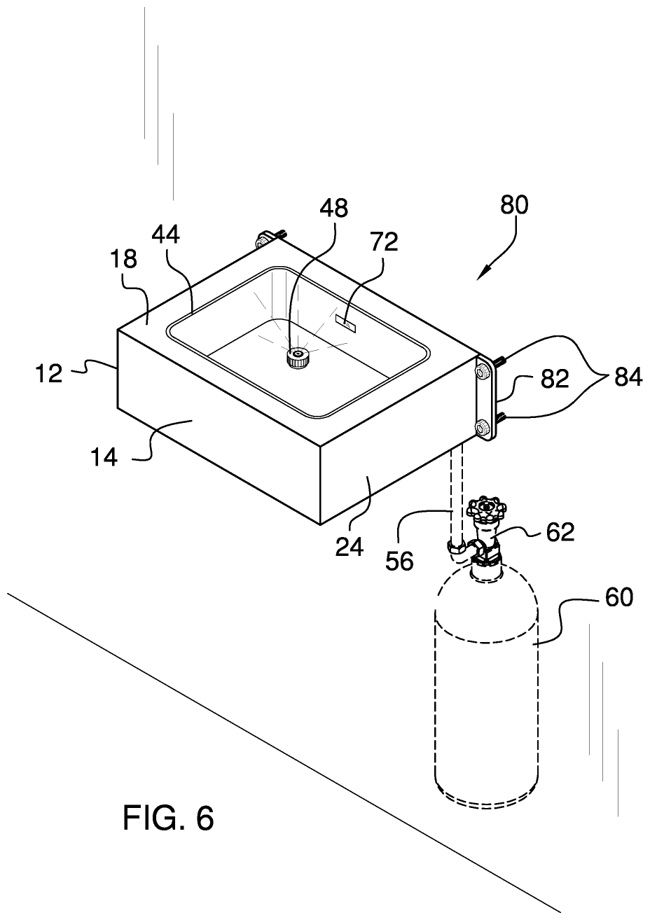

In an alternative embodiment 80 shown in FIGS. 5 and 6, the housing 12 may be rectangular prismatic. A pair of flanges 82 is coupled to the housing left side 22 and the housing right side 24 adjacent the housing back side 16. A plurality of mounting bolts 84 extends through the pair of flanges 82 and is configured to mount the housing 12 to a wall 86. The tank 60 and the release valve 68 may rest on the other side of the wall 86.

In use, the user places his or her hands within the basin 32. The motion sensor 72 then activates the release valve to allow flow of the pressurized hand sanitizer from the tank 60 to come through the misting tip 48 and create a mist of hand sanitizer. The user then passes his or her hands through the mist to sanitize them without needing to rub them together for full coverage.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A hand sanitizing station apparatus comprising:
a housing having a housing front side, a housing back side, a housing top side, a housing bottom side, a housing left side, and a housing right side defining a housing inside;
a basin coupled to the housing, the basin being coupled to the housing top side and having a basin front side, a basin back side, a basin left side, a basin right side, a basin bottom side, and a basin rim, the basin bottom side having a tip aperture extending therethrough;
a misting tip coupled to the basin, the misting tip having a stem extending through the tip aperture and a head coupled to the stem above the basin bottom side, the head having a release aperture to create a mist;
a feeder tube coupled to the misting tip, the feeder tube being in fluid communication with the stem of the misting tip;
a tank coupled to the feeder tube, the tank containing pressurized hand sanitizer;
a release valve coupled to the feeder tube, the release valve being coupled between the tank and the misting tip; and
a motion sensor coupled to the basin, the motion sensor being in operational communication with the release valve to allow flow of the pressurized hand sanitizer through to the release aperture of the misting tip when a user's hands are placed within the basin.

2. The hand sanitizing station apparatus of claim 1 further comprising the basin rim being coupled flush with the housing top side.

3. The hand sanitizing station apparatus of claim 1 further comprising the tank being coupled within the housing inside.

4. The hand sanitizing station apparatus of claim 3 further comprising the housing top side having an upper tier and a lower tier; there being a pair of basins including an upper basin and a lower basin coupled to the upper tier and the lower tier, respectively; a pair of misting tips coupled to the pair of basins; a pair of feeder tubes coupled to the pair of misting tips; a pair of release valves coupled to the pair of feeder tubes; and a pair of motion sensors coupled to the pair of basins.

5. The hand sanitizing station apparatus of claim 3 further comprising the housing back side having an access door.

6. The hand sanitizing station apparatus of claim 1 further comprising a pressure regulator coupled to the tank.

7. The hand sanitizing station apparatus of claim 6 further comprising the pressure regulator including a pressure gauge and a spurred handle.

8. The hand sanitizing station apparatus of claim 1 further comprising the housing being rectangular prismatic; a pair of flanges being coupled to the housing left side and the housing right side adjacent the housing back side; and a plurality of mounting bolts extending through the pair of flanges, the plurality of mounting bolts being configured to mount the housing to a wall.

9. A hand sanitizing station apparatus comprising:
a housing having a housing front side, a housing back side, a housing top side, a housing bottom side, a housing left side, and a housing right side defining a housing inside, the housing top side having an upper tier and a lower tier, the housing back side having an access door;
a pair of basins coupled to the housing, the pair of basins including an upper basin and a lower basin coupled to the upper tier and the lower tier, respectively, each basin having a basin front side, a basin back side, a basin left side, a basin right side, a basin bottom side, and a basin rim, the basin rim being flush with the housing top side, the basin bottom side having a tip aperture extending therethrough;
a pair of misting tips coupled to the pair of basins, each misting tip having a stem extending through the tip aperture and a head coupled to the stem above the basin bottom side, the head having a release aperture to create a mist;
a pair of feeder tubes coupled to the pair of misting tips, the feeder tube being in fluid communication with the stem of the misting tip;
a tank coupled to the feeder tube, the tank containing pressurized hand sanitizer, the tank being coupled within the housing inside;
a pressure regulator coupled to the tank, the pressure regulator including a pressure gauge and a spurred handle;
a pair of release valves coupled to the pair of feeder tubes, each release valve being coupled between the tank and the respective misting tip; and
a pair of motion sensors coupled to the pair of basins, each motion sensor being in operational communication with the respective release valve to allow flow of the pressurized hand sanitizer through to the release aperture of the respective misting tip when a user's hands are placed within the respective basin.

10. A hand sanitizing station apparatus comprising:
a housing having a housing front side, a housing back side, a housing top side, a housing bottom side, a housing left side, and a housing right side defining a housing inside, the housing being rectangular prismatic;
a pair of flanges coupled to the housing, the pair of flanges being coupled to the housing left side and the housing right side adjacent the housing back side;
a plurality of mounting bolts extending through the pair of flanges, the plurality of mounting bolts being configured to mount the housing to a wall;
a basin coupled to the housing, the basin being coupled to the housing top side and having a basin front side, a basin back side, a basin left side, a basin right side, a basin bottom side, and a basin rim, the basin bottom side having a tip aperture extending therethrough;
a misting tip coupled to the basin, the misting tip having a stem extending through the tip aperture and a head coupled to the stem above the basin bottom side, the head having a release aperture to create a mist;
a feeder tube coupled to the misting tip, the feeder tube being in fluid communication with the stem of the misting tip;
a tank coupled to the feeder tube, the tank containing pressurized hand sanitizer;
a pressure regulator coupled to the tank, the pressure regulator including a pressure gauge and a spurred handle;
a release valve coupled to the feeder tube, the release valve being coupled between the tank and the misting tip; and
a motion sensor coupled to the basin, the motion sensor being in operational communication with the release valve to allow flow of the pressurized hand sanitizer through to the release aperture of the misting tip when a user's hands are placed within the basin.
